(19) United States
(12) Patent Application Publication
(10) Pub. No.: US 2006/0177529 A1
(43) Pub. Date: Aug. 10, 2006

(54) Aleurone product and corresponding production method

(75) Inventors: Josef Laux, Ermatingen (CH); Walter Von Reding, Sattel (CH); Gaspard Widmer, Uzwil (CH)

Correspondence Address: GREENBLUM & BERNSTEIN, P.L.C., 1950 ROLAND CLARKE PLACE, RESTON, VA, 20191, US

(73) Assignee: BUEHLER AG, Uzwil (CH)

(21) Appl. No.: 10/546,254

(22) PCT Filed: Jan. 13, 2004
(86) PCT No.: PCT/CH04/00012

§ 371 Date: Aug. 19, 2005

(30) Foreign Application Priority Data
Feb. 20, 2003 (DE) 103 07 401.5
May 5, 2003 (DE) 103 20 076.2

(51) Int. Cl. A61K 36/899; C12N 5/04

(52) U.S. Cl. 424/750; 435/419

(57) ABSTRACT

The invention relates to an aleurone product comprising particles originating from the aleurone cells (of cereal, in particular the aleurone cells of wheat. The invention is characterised in that the particles originating from the cereal aleurone cells are finely ground with a diameter of up to 500 μm as the longest dimension. The inventive aleurone product is produced as follows: preparation of bran, in particular wheat bran, which consists of aleurone constituents and non-aleurone constituents: separation of the aleurone constituents from the non-aleurone constituents to obtain a blend of alcurone constituents and non-aleurone constituents: adjustment of the humidity of the blend to achieve a value of 10-20 wt % water; and fine grinding (micro-grinding) of the blend by means of a friction roller mill comprising at least one roller pair, whose rollers are operated with different surface speeds and are pressed against one another.

The invention relates to an aleuron product, which has particles consisting of grain aleuron cells, in particular wheat aleuron cells, as well as to a method for its manufacture.

Aleuron is a nutritionally extremely valuable component of grain, in particular of wheat grain. Aleuron is present in wheat grain as a single-cell layer (aleuron layer) between the flour body (endosperm) and shell (pericarp and testa) of the wheat grain. The percent by weight of aleuron in the wheat averages 8.3%.

Aleuron is isolated from bran and further processed using physical, in particular mechanical-abrasive and biological-enzymatic methods. The bran used as the parent material for this purpose is obtained in a conventional manner in a grain mill. In this regard, reference is made to International Patent Application WO 01/15711 A2 of the same applicant.

The aleuron cells of the wheat grain contain the most important nutritional substances of the wheat grain in concentrated form, such as vitamins, minerals, essential fatty acids, nutrient fibers, high-quality protein (albumin), along with special protective substances (bioactive substances, such as polyphenols, lignan, phytin, etc.). Wheat has also been a proven foodstuff for humans and animals for several thousand years.

The high content of vitamins and minerals contribute to overall health, as well as to mental and physical performance.

The nutrient fibers in the aleuron help improve digestion in the large intestine (prebiotic). In addition, ion exchange activity slows resorption in the small intestine, and binds undesired substances. In so doing, they contribute to a long-lasting feeling of satisfaction and detoxification.

Bioactive substances of the aleuron, such as polyphenols, flavonoids, lignan, beta-glucan, etc., help protect against several illnesses afflicting human civilization, such as arterial disease and certain forms of cancer.

The object of the invention is to make aleuron and its constituents useful for human and animal nutrition.

The object is achieved according to the invention by virtue of the fact that the aleuron product mentioned at the outset has finely milled particles consisting of grain aleuron cells, having a maximum length ranging from about one aleuron diameter to 500 μm. In this finely milled form, these aleuron-containing particles can be used in an especially wide variety of ways for human and animal nutrition, in particular as additives to conventional foodstuffs. The longest measure preferably ranges from one aleuron cell diameter to 500 μm in more than 50% of the particles consisting of aleuron cells.

An even finer aleuron product is preferred for certain applications. The longest measure can range from 100-300 μm. The longest measure preferably ranges from 100-300 μm in more than 50% of the particles consisting of aleuron cells. The longest measure can also range from 10-100 μm, wherein the longest measure preferably ranges from 10-100 μm in more than 50% of the particles consisting of aleuron cells here as well.

According to the invention, the particles consisting of aleuron cells contain cell clusters made up of 1 to 200 aleuron cells, wherein preferably more than 50% of the particles consisting of aleuron cells are cell clusters made up of 1 to 200 aleuron cells.

In the somewhat finer aleuron product, the particles consisting of aleuron cells have cell clusters made up of 5 to 100 aleuron cells, wherein preferably more than 50% of its particles consisting of aleuron cells are cell clusters made up of 5 to 100 aleuron cells.

One special property of the aleuron product according to the invention aid that the smallest dimension of the particles consisting of grain aleuron cells roughly corresponds to the size of a single aleuron cell. In other words, the individual cells of the generally single-cell aleuron layer (wheat) are still intact. The fine milling and subsequent screening only determined the size or number of aleuron cells of the cell groups also referred to as clusters.

Even though aleuron is also contained in the seeds of other grain types, use is preferably made of the aleuron cells from wheat bran.

The bioavailability of the substances contained in the aleuron cells and mentioned at the outset depends greatly on the condition of the cell walls of the aleuron cells in the cell clusters according to the invention.

In a special embodiment, the cell walls of all aleuron cells of the particles are undamaged. This can be achieved in a gentle fine milling process. The advantage to this is that the aleuron cell content can be retained over a long period of time. Primarily, the cell constituents are prevented from oxidizing.

In an alternative embodiment, a portion of the aleuron cells of the particles have damaged cell walls. This makes the valuable constituents of the aleuron cells consisting primarily of arabinoxylanes more accessible during digestion than in the case of intact aleuron cells. In order to prevent the cell contents from prematurely oxidizing, various antioxidants can be mixed into the aleuron product according to the invention as needed.

The cell walls of the aleuron cells can be at least partially damaged or weakened enzymatically, chemically, mechanically or thermally. Also possible is a combination of these measures, e.g., a mechanical-enzymatic or mechanical-thermal weakening of the cell walls. Depending on the extent of damage/weakening of the aleuron cell walls, humans or animals can absorb the nutritionally valuable substances more quickly or completely, or perhaps more slowly and incompletely, during the digestive process, wherein the prebiotic effect alone dominates in the latter case due to the cells present in the digestive tract. Specifically adjusting the level of damage to the aleuron cells makes it possible to set the rate at which the nutritionally valuable substances are released in a targeted fashion. Highly damaged aleuron cells lead to a quick and virtually complete digestion of cell contents, while the digestion (fermentation) of slightly damaged cells takes more time.

In a special embodiment, the aleuron product is a mixture consisting of a first standard aleuron product and a second standard aleuron product, wherein it is preferred that first standard aleuron product have primarily undamaged aleuron cells, and the second standard aleuron product have primarily damaged aleuron cells. This makes it possible to manufacture a “tailored” aleuron product that can be adjusted to the physiological requirements of specific individuals (humans or animals).

The second standard aleuron has primarily destroyed aleuron cells, or consist primarily of aleuron cell fragments.

The aleuron product according to the invention preferably has the following composition: 10-30% w/w protein, 5-15% w/w ash, 2-8% w/w fat and 30-60% w/w indigestible or only partially digestible fibers.

In one significant achievement of this invention, the aleuron product according to the invention has more than 90% w/w aleuron, in particular more than 95% w/w aleuron. A practically pure “aleuron powder” an also be prepared according to the invention. Given a product according to the invention based on wheat bran, practically no outer shell particles that would cause the product to turn brown are present any longer. Rather, this highly pure “aleuron powder” according to this invention is approximately white or yellowish to white, and it exhibits practically no dark brown shell particles.

In another embodiment, the aleuron product according to the invention can have shell particles still stemming from the original bran in addition to aleuron cell clusters. This product can be manufactured somewhat more easily and cost-effectively.

The method for manufacturing the aleuron product according to the invention involves the following steps:
a) Preparation of bran, in particular wheat bran, which consists of aleuron constituents and non-aleuron constituents;
b) Detachment of aleuron constituents from the non-aleuron constituents, thereby producing a mixture consisting of aleuron constituents and non-aleuron constituents;
c) Setting of mixture moisture to a value of 10-20% w/w water;
d) Super-fine milling of the mixture using a distributing roll mill with at least one roll pair, the rolls of which are operated at different surface velocities and pressed against each other, exposing the aleuron constituents to compressive and shearing forces.

Placing a mechanical load on the aleuron particles in step d) makes the nutritionally valuable contents of the aleuron cells more easily accessible for digestion in the individual, and changes the composition (water removal) and taste (debitterizing) of the aleuron product.

In step c), the moisture is preferably set to 14-16% w/w water. Setting the moisture of the sorted aleuron constituents to a value of 10-20% w/w, in particular to 14-16% w/w water, has a positive effect, since is surprisingly improves the taste, primarily debitterizes, the aleuron when combined with the subsequent fine milling.

It is particularly advantageous that step d) be preceded by a step to sort the aleuron constituents out of the mixture. This is because, if too many non-aleuron constituents, such as shell particles, are contained in the mixture to be supplied to fine milling, the relatively hard foreign particles hamper fine milling, practically acting as “spacers” between the friction rolls, thereby protecting the actual aleuron particles (=aleuron cell clusters) against comminution by the rolls.

This sorting process is preferably performed electrostatically, preferably before step c). Electrostatically sorting the aleuron constituents makes it possible to obtain practically pure “aleuron powder”, which can not only be milled even finer, but even strictly visually more resembles a yellowish-white powder than the brownish, finely milled product contaminated with shell particles.

During fine milling, the aleuron constituents are best passed through a roll pair for repeated friction milling, or the aleuron constituents are passed through several sequential roll pairs for repeated friction milling, wherein the roll gap is set practically to zero, and the rolls are pressed together.

The fine milling step is preferably followed by another step for sorting, preferably using a method for the wet separation of the finely milled (micromilled) aleuron constituents. This makes it possible to obtain the mentioned fragments in a range of 50-500 μm, in a range of 100-300 μm, or in a range of 10-100 μm.

Additional advantages, features and possible applications are set forth in the following non-limiting description of preferred exemplary embodiments of the aleuron product based on the drawing, wherein:

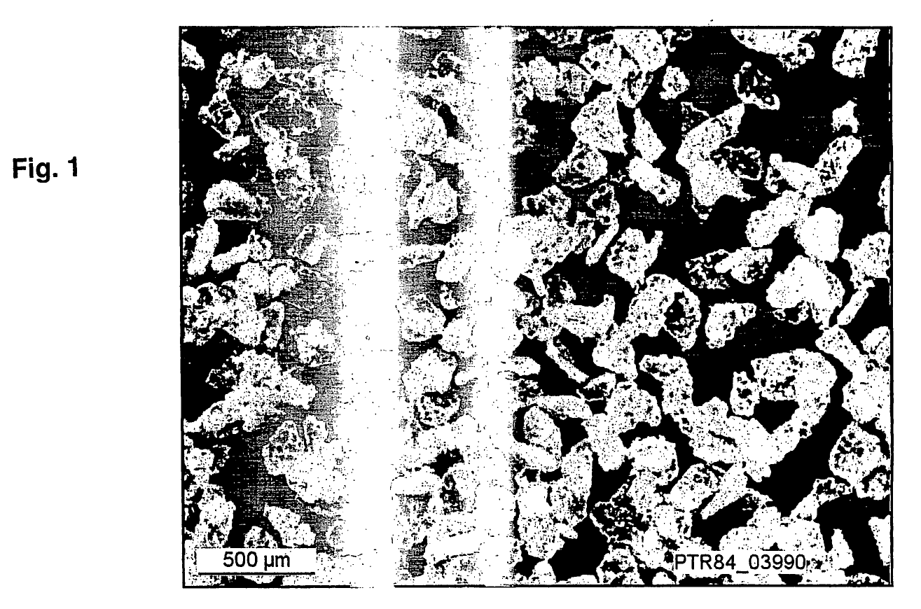

FIG. 1 shows a microscopic image of primarily undamaged aleuron cell clusters according to the invention;

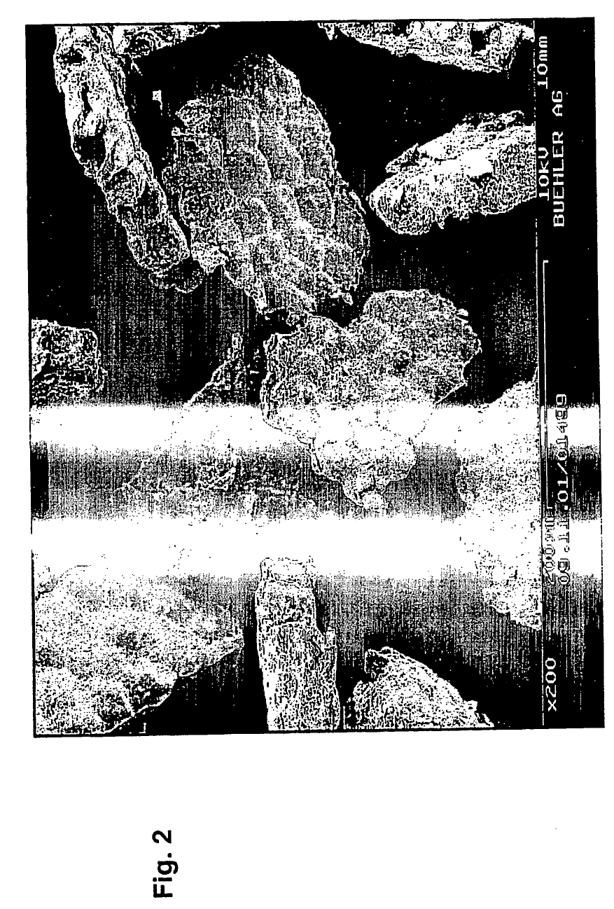

FIG. 2 shows an electron microscope image of the primarily undamaged aleuron cell clusters of FIG. 1, at a higher resolution;

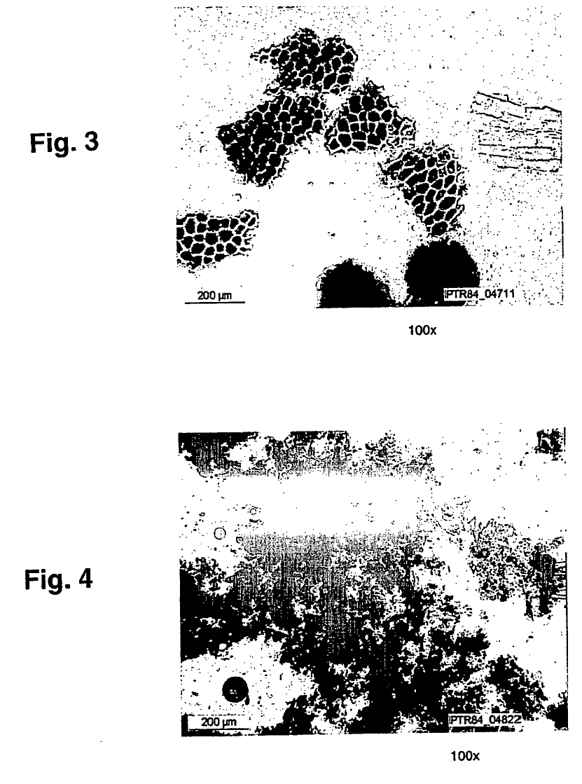

FIG. 3 is a microscope image showing a mixture according to the invention of primarily undamaged aleuron cell clusters (“aleuron particles”) and shell particles;

FIG. 4 a microscope image showing a mixture, according to the invention, of hardly any undamaged aleuron cell clusters (aleuron particles”), but rather consisting primarily of undamaged aleuron cell clusters (“aleuron sludge”).

FIG. 1 shows a microscopic image of primarily undamaged aleuron cell clusters 1, as are typically present after step b) of the method according to the invention. The aleuron cell clusters 1 shown here each consist of a single-cell layer of aleuron cells 2 (see FIG. 2). These clusters each represent fragments, which stem from the single-cell aleuron layer between the shell and the endosperm of a wheat grain. The figure shows a fragment in which the longest measure of the aleuron cell clusters 1 ranges from about 100-300 μm.

FIG. 2 shows an electron microscopic image of the primarily undamaged aleuron cell composites 1 of FIG. 1, at a higher resolution. The individual aleuron cells 2 and the cell walls 3 are now clearly discernible. The overall few damaged aleuron cells 2 are concentrated at the edge of the platelet-like aleuron cell cluster 1, where the original, integral single-cell aleuron layer of a wheat grain was typically broken apart in steps a) and b). The cell walls 3 of the aleuron cells 2 primarily consist of arabinoxylanes, and are difficult if not impossible to break down or “crack” while being digested in humans and many animals. However, they are slowly fermented by bacteria in the digestive tract. The initially mentioned and other nutritionally valuable constituents are therefore made available to the organism of the digesting human or animal to only a limited extent.

FIG. 3 is a macroscopic image showing a mixture according to the invention of primarily undamaged aleuron cell clusters (“aleuron particles”) and shell particles. Also visible in addition to the total of five aleuron cell clusters 1 is a shell particle 4.

FIG. 4 is a macroscopic image showing a mixture according to the invention consisting of aleuron cell clusters that are now hardly undamaged (“aleuron particles”), but rather consist of primarily damaged aleuron cell clusters (“aleuron sludge”). The damaged aleuron cell clusters 5, in which a majority of aleuron cells 2 (see FIG. 2) have broken open or burst, and whose cell contents have partially spilled out, now form an “aleuron sludge”, i.e., a mixture of a few cell clusters 1 each now having only a very few aleuron cells 2, individual aleuron cells 2, damaged cell clusters and damaged aleuron cells, and leaked cell contents (“aleuron juice”) of the damaged aleuron cells. The damaged or destroyed cell walls 3 of the aleuron cells 2 consisting primarily of arabinoxylanes only release a majority of initially mentioned and additional nutritionally valuable constituents of the aleuron cells, thereby now making them available for digestion in humans and many animals, without losing the prebiotic effect of the actual cell walls in the large intestine (fermentation of arabinoxylanes).

The bioavailability of nutritionally valuable constituents in the wheat grain increases for the following forms of ingestion for aleuron:

Whole wheat grain (whole grain)

Shell removed from wheat grain, with adhering aleuron layer (whole bran)

Mixture of “aleuron particles” and “shell particles” (comminuted bran)

Isolated “aleuron particles” (only aleuron cell clusters remain)

Super-finely milled aleuron particles (“aleuron sludge”)

While the increase in bioavailability from 1) to 2), 2) to 3) and 3) to 4) is relatively small, a significant increase in bioavailability could be achieved via super-fine milling according to the invention.

This is illustrated by Table 1: LEURON®, which shows a quantitative comparison between the aleuron ingestion forms 3), 4) and 5).

TABLE 1
LEURON ®

| Analysis per 100 g | Method | Units | 3) | 4) | 5) |
|---|---|---|---|---|---|
| Calories | Calculation | cal | 173 | 202 | 236 |
| Fat, total | AACC 30-14 | g | 6.8 | 8.0 | 8.8 |
| Ash | AACC 08-01 | g | 8.5 | 10.6 | 10.7 |
| Carbohydrate, total | Calculation | g | 59.0 | 53.0 | 52.4 |
| Nutrient fibers, total (NFT) | AACC 32-07 | g | 49.6 | 42.8 | 36.8 |
| Insoluble nutrient fiber | AACC 32-07 | g | 46.6 | 38.8 | 32.5 |
| Percent of NFT | AACC 32-07 | % | 94 | 91 | 88 |
| Soluble nutrient fiber | AACC 32-07 | g | 3.1 | 4.1 | 4.3 |
| Percent of NFT | AACC 32-07 | % | 6 | 9 | 12 |
| Protein (Factor 5.7) | AACC 46-12 | g | 15.6 | 18.2 | 19.3 |
| Moisture | AACC 44-15A | g | 10.1 | 10.2 | 8.8 |

Key:
3) Mixture of aleuron particles and shell particles
4) Pure aleuron particle powder
5) Super-finely milled aleuron particles

Table 2 shows additional typical compositions for the aleuron preparations according to the invention.

TABLE 2
Typical Compositions of Aleuron Preparations

| | Method | Unit | ASP-1 | ASP-2 |
|---|---|---|---|---|
| Crude protein | Leco | g/100 g DM | 16.5 | 20.8 |
| (Nx5.70) | | | | |
| Crude fat | Sokhlet | g/100 g DM | 5.8 | 5.7 |
| polyunsaturated | HPLC-fatty | % of crude | 66 | 66 |
| fatty acids | acid | fat | | |
| | spectrum | | | |
| monounsaturated | | | 18 | 18 |
| fatty acids | | | | |
| saturated fatty | | | 16 | 16 |
| acids | | | | |
| Nutrient fibers, | AOAC 991.43 | g/100 g DM | 54.1 | 47.1 |
| total | | | | |
| water insoluble | | g/100 g DM | 50.0 | 43.0 |
| water soluble | | g/100 g DM | 4.1 | 4.1 |
| Crude ash | Incineration | g/100 g DM | 9.3 | 11.3 |
| phosphorus | furnace/590° C. | g/kg DM | 21.2 | 25.8 |
| potassium | | g/kg DM | 18.5 | 22.5 |
| magnesium | Magnesium | g/kg DM | 8.4 | 10.3 |
| calcium | mineralization + | mg/kg DM | 762 | 930 |
| iron | AAS | mg/kg DM | 213 | 260 |
| zinc | | mg/kg DM | 114 | 139 |
| sodium | | mg/kg DM | 17 | 21 |
| Vitamins | Swiss man- | mg/100 g DM | n.a. | 1.4 |
| B1 (thiamin) | ual of | mg/100 g DM | n.a. | 0.2 |
| B2 (riboflavin) | food ana- | mg/100 g DM | n.a. | 1.3 |
| niacin | lysis (266) | mg/100 g DM | n.a. | 32.9 |
| folic acid | | μg/100 g DM | n.a. | 158 |
| panthothenic | | mg/100 g DM | n.a. | 4.9 |
| acid | | | | |
| E (DL-α-toco- | | mg/100 g DM | n.a. | 1.2 |
| pherol-AC0 | | | | |
| Phytinic acid | Egli (267) | g/100 g DM | 6.9 | 8.4 |

Key:
ASP-1: Aleuron product, manufactured as follows:
Wheat bran containing aleuron and non-aleuron constituents was used as the basis.
The aleuron constituents were mechanically detached from the non-aleuron constituents, thereby yielding a mixture of aleuron constituents and non-aleuron constituents.
The moisture of the mixture was then set to a value of 10-20% w/w water.
The mixture prepared in this way was subjected to super-fine milling (micro-milling) with a distributing roll mill having at least one roll pair. The rolls were operated at varying surface velocities, and pressed against each other.
The resultant product, ASP-1, contains shell particles in addition to aleuron particles (clusters of aleuron cells).
ASP-2: Aleuron product, manufactured as follows
ASP-1 was taken as the basis.
The ASP-1 was enriched to increase the share of aleuron particles.

The sorting of shell particles required for this purpose is accomplished by contact-electrically or triboelectrically charging ASP-1 and then electrostatically separating the shell particles from the aleuron particles.

The resultant product, ASP-1, has a high aleuron particle content.

### REFERENCE LIST

1 Undamaged aleuron cell cluster (“aleuron particles”)
2 Aleuron cell
3 Cell wall
4 Shell particles
5 Damaged aleuron cell cluster (“aleuron sludge”)

1-39. (canceled)

40. An aleuron product, comprising particles of grain aleuron cells, said particles comprising finely milled particles having a maximum dimension ranging from one aleuron cell diameter to 500 μm, said particles comprising aleuron cells are clusters, each cluster comprising 1 to 200 aleuron cells.

41. The aleuron product according to claim 40, wherein the maximum dimension ranges from 100-300 μm.

42. The aleuron product according to claim 40, wherein the maximum dimension ranges from 10-100 μm.

43. The aleuron product according to claim 40, wherein the particles comprising aleuron cells are cell clusters made up of 5 to 100 aleuron cells.

44. The aleuron product according to claim 40, wherein a smallest dimension of the particles comprising grain aleuron cells corresponds to roughly a size of a single aleuron cell.

45. The aleuron product according to claim 40, wherein the particles coming from grain aleuron cells are in each case formed through a unicellular layer from aleuron cells.

46. The aleuron product according to claim 40, wherein the aleuron cells originate from wheat bran.

47. The aleuron product according to claim 40, wherein the cell walls of all aleuron cells of particles are undamaged.

48. The aleuron product according to claim 40 wherein a portion of the aleuron cells of the particles have damaged cell walls.

49. The aleuron product according to claim 48, wherein the product comprises a mixture of a first standard aleuron product and a second standard aleuron product.

50. The aleuron product according to claim 49, wherein the first standard aleuron product has predominantly undamaged aleuron cells, and the second standard aleuron product has predominantly damaged aleuron cells.

51. The aleuron product according to claim 50, wherein the second standard aleuron product has predominantly destroyed aleuron cells, or predominantly composed of aleuron cell fragments and leaked cell contents.

52. The aleuron product according to claim 51, wherein the aleuron product is a molding.

53. The aleuron product according to claim 48, wherein the damaged cell walls are enzymatically weakened cell walls.

54. The aleuron product according to claim 48, wherein the damaged cell walls are mechanically weakened cell walls.

55. The aleuron product according to claim 40, wherein the product contains 10-30 wt % protein.

56. The aleuron product according to claim 40, wherein the product contains 5-15 wt % ash.

57. The aleuron product according to claim 40, wherein the product contains 2-8 wt % fat.

58. The aleuron product according to claim 40, wherein the product has 30-60 wt % indigestible or only partially digestible fibers.

59. The aleuron product according to claim 40, wherein the product has more than 95 wt % aleuron.

60. The aleuron product according to claim 40, wherein the product has more than 99 wt % aleuron.

61. The aleuron product according to claim 60, wherein the product has hull particles in addition to aleuron cell clusters.

62. The aleuron product according to claim 40, wherein the aleuron cells comprise wheat aleuron cells.

63. A method for manufacturing an aleuron product, comprising:
a) preparing bran comprising aleuron constituents and non-aleuron constituents;
b) detaching aleuron components from the non-aleuron components, thereby producing a mixture composed of aleuron constituents and non-aleuron constituents;
c) setting of the mixture moisture to a value of 10-20 wt % water;
d) superfine milling (micro-milling) the mixture using a grinding roll mill with at least one roll pair, the rolls of which are operated at different surface velocities and pressed against each other.

64. The method according to claim 63, wherein the moisture in c) is set to 14-16 wt % water.

65. The method according to claim 63, wherein d) is preceded by sorting the aleuron constituents out of the mixture.

66. The method according to claim 65, wherein the sorting out takes place electrostatically.

67. The method according to claim 63, wherein in d) the aleuron constituents are passed through a roll pair several times for repeated friction milling.

68. The method according to claim 63, wherein in d) the aleuron constituents are passed several times through a plurality of sequential roll pairs several times for repeated friction milling.

69. The method according to claim 63, wherein d) is followed by sorting out the finely milled (micro-milled) aleuron constituents.

70. The method according to claim 69, wherein a fraction of grain aleuron cells is sorted out whose longest dimension ranges from one aleuron cell diameter to 500 μm.

71. The method according to claim 69, wherein a fraction of grain aleuron cells is sorted out whose longest dimension ranges from 100-300 μm.

72. The method according to claim 69, wherein a fraction of grain aleuron cells is sorted out whose longest dimension ranges from 10-100 μm.
